(19) United States
(12) Patent Application Publication
(10) Pub. No.: US 2026/0041402 A1
(43) Pub. Date: Feb. 12, 2026

(54) DETECTION DEVICE FOR CALCIFICATION

(71) Applicants: EDGECARE INC., Seoul (KR); SOGANG UNIVERSITY RESEARCH & BUSINESS DEVELOPMENT FOUNDATION, Seoul (KR)

(72) Inventors: Seong Jun PARK, Uiwang-si (KR); Ji Hun JANG, Seoul (KR); Il Seob SONG, Goyang-si (KR); Yang Mo YOO, Seoul (KR)

(21) Appl. No.: 19/284,886

(22) Filed: Jul. 30, 2025

(30) Foreign Application Priority Data
Aug. 12, 2024 (KR) 10-2024-0107352

(51) Int. Cl. A61B 8/00; A61B 8/08

(52) U.S. Cl. CPC A61B 8/5207; A61B 8/085; A61B 8/0891; A61B 8/488; A61B 8/5215; A61B 8/54

(57) ABSTRACT

A detection device for calcification according to an embodiment of the present disclosure may include a frequency determination unit, an ultrasound transceiver unit, and a band signal processing unit. The frequency determination unit may determine a transmission frequency of a transmitted ultrasound signal. The ultrasound transceiver unit may transmit the transmitted ultrasound signal to an object and receive a received ultrasound signal reflected from the object. The band signal processing unit may filter the received ultrasound signal to extract a calcification signal corresponding to a calcification included in the object. The detection device for calcification according to the present disclosure transmits a transmitted ultrasound signal at a frequency within a band of the ultrasound probe and includes a band signal different from a transmitted signal frequency among smoothed signals reflected from the calcification in a reception bandwidth of the ultrasound probe, thereby more accurately detecting the calcification within the object.

### BACKGROUND

### 1. Field

The present disclosure relates to a detection device for calcification.

### 2. Description of Related Art

In order to overcome the low resolution of existing ultrasound medical imaging devices in diagnosing calcification inside the human body, various studies are being conducted to increase the sensitivity of calcification diagnosis by imaging a twinkling image.

### Related Art Document

### Patent Document

(Korean Patent Laid-Open Publication) No.10-2023-0094855 (Publication date, June 28, 2023)

### SUMMARY

The present disclosures are directed to providing a detection device for calcification capable of more accurately detecting the calcification within an object by transmitting a transmitted ultrasound signal at a frequency within a band of an ultrasound probe and including a band signal different from a transmitted signal frequency among smoothed signals reflected from the calcification in a reception bandwidth of the ultrasound probe.

In order to solve the above problems, a detection device for calcification according to an embodiment of present disclosure may include a frequency determination unit, an ultrasound transceiver unit, and a band signal processing unit. The frequency determination unit may determine a transmission frequency of a transmitted ultrasound signal. The ultrasound transceiver unit may transmit the transmitted ultrasound signal to an object and receive a received ultrasound signal reflected from the object. The band signal processing unit may filter the received ultrasound signal to extract a calcification signal corresponding to the calcification included in the object.

The transmission frequency may be equal to or different from a center frequency of the ultrasound probe included in the ultrasound transceiver unit.

The band signal processing unit may determine a reception frequency. The reception frequency may be different from the transmission frequency.

The band signal processing unit may include a demodulation frequency control unit.

The demodulation frequency may be equal to the reception frequency.

The band signal processing unit may include a bandpass filter. The bandpass filter may filter the received ultrasound signal.

The center frequency of the bandpass filter may be equal to the reception frequency.

One end of a bandwidth of the bandpass filter may be larger or smaller than the transmission frequency of the transmitted ultrasound signal.

The detection device for calcification may further include an image display unit. The image display unit may provide an ultrasound calcification image based on the calcification signal.

The frequency determination unit may further include a transmission control unit. The transmission control unit may control the transmission frequency according to frequency characteristics of the calcification included in the ultrasound calcification image.

The band signal processing unit may further include a filter control unit. The filter control unit may control the center frequency of the bandpass filter and the bandwidth of the bandpass filter according to the resolution of the calcification included in the ultrasound calcification image.

The band signal processing unit may further include a plurality of bandpass filters having a predetermined bandwidth.

The detection device for calcification may further include a sub-image display unit. The sub-image display unit may provide the ultrasound calcification image based on a correlation value between a plurality of sub-calcification images generated based on each of the sub-filter signals obtained by filtering the received ultrasound signal using the plurality of bandpass filters.

In addition to the technical problems of present disclosure described above, other features and advantages of present disclosure are described below, or can be clearly understood by those skilled in the art from such description and description.

### BRIEF DESCRIPTION OF DRAWINGS

FIG. 1 is a diagram illustrating a detection device for calcification according to embodiments of the present disclosure.

FIG. 2 is a diagram illustrating a calcification spectrum and a blood flow spectrum generated by the detection device for calcification of FIG. 1.

FIGS. 3 and 4 are diagrams for describing an embodiment of a band signal processing unit included in the detection device for calcification of FIG. 1.

FIGS. 5 and 6 are diagrams for describing an image display unit included in the detection device for calcification of FIG. 1.

FIG. 7 is a diagram illustrating a transmission control unit included in the detection device for calcification of FIG. 1.

FIG. 8 is a diagram illustrating a filter control unit included in the detection device for calcification of FIG. 1.

FIG. 9 is a diagram illustrating another embodiment of a band signal processing unit included in the detection device for calcification of FIG. 1.

FIGS. 10 and 11 are diagrams for describing an embodiment of the detection device for calcification of FIG. 1.

FIG. 12 is a diagram illustrating a frequency control unit included in the detection device for calcification of FIG. 1.

FIG. 13 is a diagram illustrating a determination unit included in the detection device for calcification of FIG. 1.

FIG. 14 is a diagram illustrating a filter control unit included in the detection device for calcification of FIG. 1.

### DETAILED DESCRIPTION

In the specification, in adding reference numerals to components throughout the drawings, it is to be noted that like reference numerals designate like components even though components are shown in different drawings.

On the other hand, the meaning of the terms described in this specification should be understood as follows.

Singular expressions should be understood to include plural expressions unless the context clearly defines otherwise, and the scope of the rights should not be limited by these terms.

Also, it should be understood that terms such as “include” or “have” do not preclude the existence or addition possibility of one or more other features or numbers, steps, operations, components, parts, or combinations thereof.

Hereinafter, preferred embodiments of the present disclosure designed to solve the above problems will be described in detail with reference to the attached drawings.

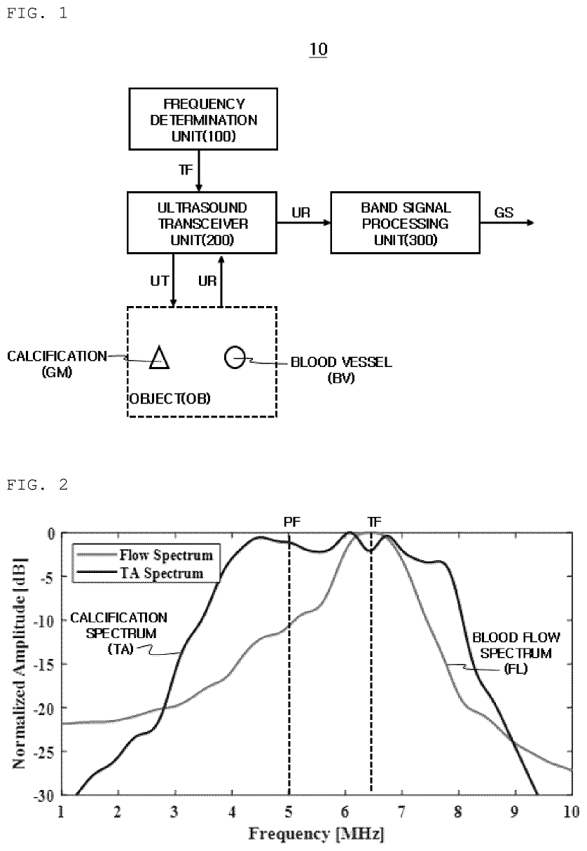

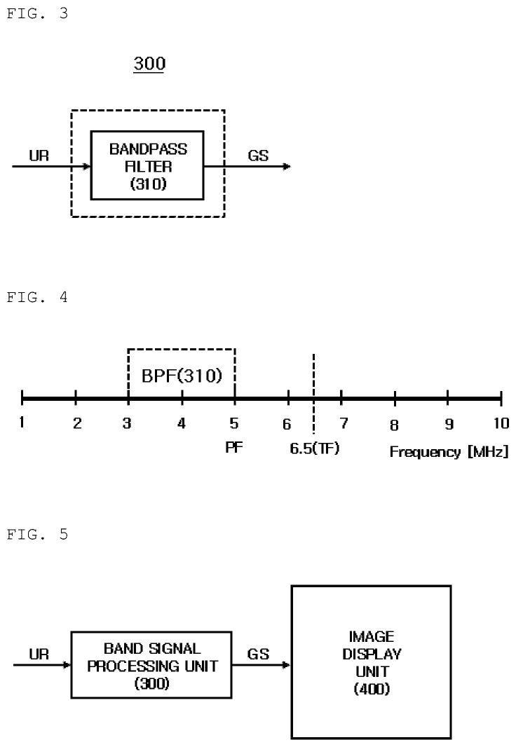

FIG. 1 is a diagram illustrating a detection device for calcification according to embodiments of the present disclosure, FIG. 2 is a diagram illustrating a calcification spectrum and a blood flow spectrum generated by the detection device for calcification of FIG. 1, and FIGS. 3 and 4 are diagrams for describing an embodiment of a band signal processing unit included in the detection device for calcification of FIG. 1.

Referring to FIGS. 1 to 4, a detection device 10 for calcification according to an embodiment of the present disclosure may include a frequency determination unit 100, an ultrasound transceiver unit 200, and a band signal processing unit 300.

The frequency determination unit 100 may determine a transmission frequency TF of a transmitted ultrasound signal UT. In an embodiment, the transmission frequency TF may be equal to or different from a center frequency PF of an ultrasound probe included in the ultrasound transceiver unit 200. For example, the center frequency PF of the ultrasound probe may be 5 MHz. When the center frequency PF of the ultrasound probe is 5 MHz, the frequency determination unit 100 may set the transmission frequency TF of the transmitted ultrasound signal UT higher than 5 MHz, which is the center frequency PF of the ultrasound probe. Here, the case where the transmission frequency TF of the transmitted ultrasound signal UT is higher than the center frequency PF of the ultrasound probe is described, but the same may be applied to the case where the transmission frequency TF of the transmitted ultrasound signal UT is lower than the center frequency PF of the ultrasound probe.

The ultrasound transceiver unit 200 may transmit the transmitted ultrasound signal UT to an object OB and receive a received ultrasound signal UR reflected from the object OB. The object OB may include not only a blood vessel BV through which blood flows, but also human tissues and calcifications formed in the human body. For example, the transmission frequency TF of the transmitted ultrasound signal UT may be 6.5 MHz, which is higher than the center frequency PF of the ultrasound probe, which is 5 MHz. The ultrasound transceiver unit 200 may transmit the transmitted ultrasound signal UT having the transmission frequency of 6.5 MHz to the object OB and receive the received ultrasound signal UR reflected from the object OB. In this case, as in the spectrum of FIG. 2, when analyzing a blood flow spectrum FL for blood flowing in the blood vessel BV and a calcification spectrum TA for calcification GM located inside the object OB using the received ultrasound signal UR, it may be seen that the blood flow spectrum FL and the calcification spectrum TA have different characteristics. More specifically, the calcification spectrum TA has a smoothed signal size in a relatively wide bandwidth compared to the blood flow spectrum FL. Here, the calcification may include calcification and limestone materials, etc., generated in the human body.

The band signal processing unit 300 may filter the received ultrasound signal UR to extract a calcification signal GS corresponding to the calcification GM included in the object OB. In an embodiment, the band signal processing unit 300 may include a bandpass filter 310. The bandpass filter 310 may filter the received ultrasound signal UR. In another embodiment, the center frequency BF of the bandpass filter 310 may be different from the center frequency PF of the ultrasound probe. For example, as in the spectrum of FIG. 2, in the band lower than the center frequency PF of the ultrasound probe, the size of the signal generated from the calcification may be high, and the size of the signal generated from the blood flow may be small. In this case, when a center frequency BF of the bandpass filter 310 is set lower than the center frequency PF of the ultrasound probe, the band signal processing unit 300 may more effectively extract the calcification signal GS from the received ultrasound signal UR. Here, the center frequency PF of the ultrasound probe may be a frequency corresponding to a center of the bandwidth of the ultrasound probe, and the center frequency BF of the bandpass filter 310 may be a frequency corresponding to a center of the bandwidth of the bandpass filter 310. Conversely, when the transmission frequency TF of the transmitted ultrasound signal UT is smaller than the center frequency PF of the ultrasound probe, the center frequency BF of the bandpass filter 310 may be set higher than the center frequency PF of the ultrasound probe.

In an embodiment, a demodulation frequency and one end of the bandwidth of the bandpass filter 310 may be larger or smaller than the transmission frequency TF of the transmitted ultrasound signal UT. For example, as illustrated in FIG. 4, the transmission frequency TF of the transmitted ultrasound signal UT may be 6.5 MHz, and the bandwidth of the bandpass filter 310 may be 2 MHz from 3 MHz to 5 MHz. In this case, one end of the bandpass filter 310 may be 5 MHz, and one end of the bandwidth of the bandpass filter 310, 5 MHz, may be smaller than the transmission frequency TF of the transmitted ultrasound signal UT, 6.5 MHz. In this way, when one end of the bandpass filter 310 is set, the calcification signal GS may be extracted more effectively from the received ultrasound signal UR. Here, the case where the transmission frequency TF of the transmitted ultrasound signal UT is higher than the center frequency PF of the ultrasound probe is described, but the same may be applied to the case where the transmission frequency TF of the transmitted ultrasound signal UT is lower than or equal to the center frequency PF of the ultrasound probe.

Also, although a specific band of the calcification signal smoothed by the bandpass filter is imaged here, the smoothed calcification signal may be included in a reception band by making the demodulation frequency different from the transmission frequency.

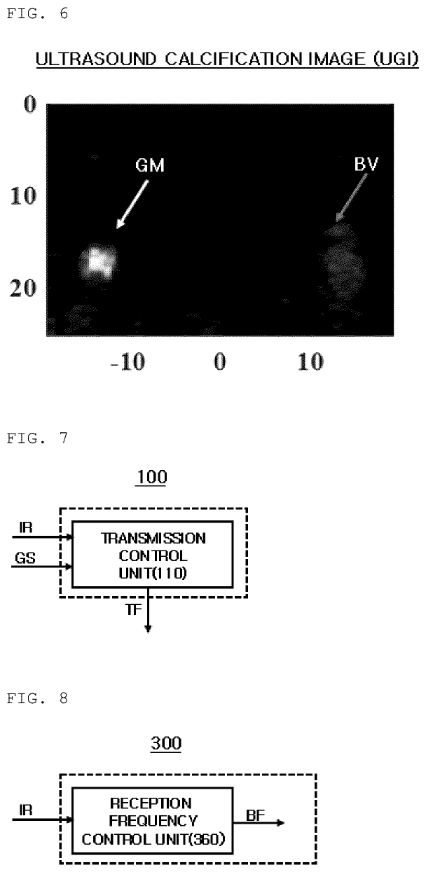

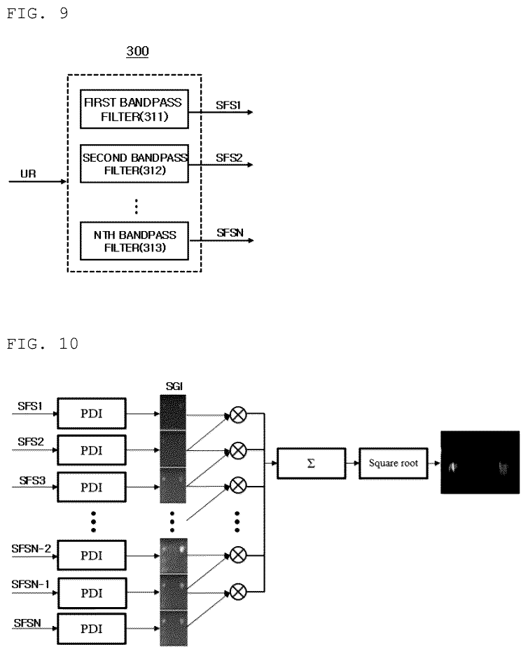

FIGS. 5 and 6 are diagrams for describing an image display unit included in the detection device for calcification of FIG. 1, FIG. 7 is a diagram illustrating a transmission control unit included in the detection device for calcification of FIG. 1, and FIG. 8 is a diagram illustrating a filter control unit included in the detection device for calcification of FIG. 1.

Referring to FIGS. 1 to 8, in an embodiment, the detection device 10 for calcification may further include an image display unit 400. The image display unit 400 may provide an ultrasound calcification image UGI based on the calcification signal GS. For example, a method of implementing an ultrasound calcification image UGI using a calcification signal GS may be the same as a method of implementing an ultrasound image using a conventional Doppler signal. In this case, as illustrated in FIG. 6, not only the calcification GM but also a blood flow image for blood flowing through the blood vessel BV may be expressed based on the calcification signal GS, but the detection device 10 for calcification according to the present disclosure may implement the calcification GM more clearly than the conventional method. For reference, when the calcification GM is disposed inside the object OB, the signal reflected from the calcification GM by the transmitted ultrasound signal UT has a random phase, and when the signal reflected from the calcification GM is imaged, the signal reflected from the calcification GM may be expressed as a sparkling virtual signal like blood flow.

In an embodiment, the frequency determination unit 100 may further include a transmission control unit 110. The transmission control unit 110 may control the transmission frequency TF according to the frequency characteristics of the calcification GM included in the ultrasound calcification image UGI. For example, when smoothing of a low-band signal of the calcification GM included in the ultrasound calcification image UGI is excellent, the transmission control unit 110 may increase the transmission frequency TF.

In an embodiment, the band signal processing unit 300 may further include a reception frequency control unit 360. The reception frequency control unit 360 may also control the demodulation frequency, the center frequency BF of the bandpass filter 310, and the bandwidth of the bandpass filter 310 according to the resolution IR of the calcification GM included in the ultrasound calcification image UGI.

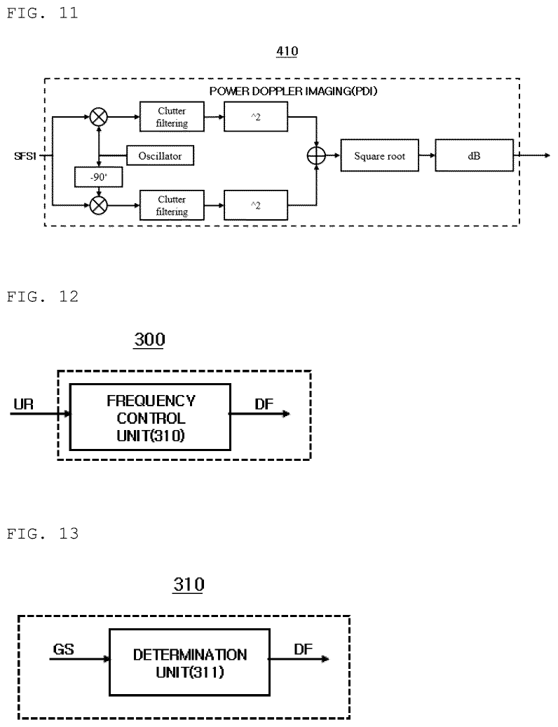

FIG. 9 is a diagram illustrating another embodiment of the bandpass signal processing unit included in the detection device for calcification of FIG. 1, and FIGS. 10 and 11 are diagrams for describing an embodiment of the detection device for calcification of FIG. 1.

Referring to FIGS. 1 to 11, in an embodiment, the band signal processing unit 300 may further include a plurality of bandpass filters having a predetermined bandwidth.

In an embodiment, the detection device 10 for calcification may further include a sub-image display unit 410. The sub-image display unit 410 may provide the ultrasound calcification image UGI based on a correlation value between a plurality of sub-calcification images generated based on each of the sub-filter signals that filter the received ultrasound signal UR using the plurality of bandpass filters. For example, the plurality of bandpass filters may include a first bandpass filter 311 to an Nth bandpass filter 313. Each of the bands of the first to Nth bandpass filters 311 to 313 may be disposed in a band lower than the center frequency PF of the ultrasound probe. When the received ultrasound signal UR is filtered using the first to Nth bandpass filters 311 to 313, first to Nth sub-filter signals SFS1 to SFSN may be obtained. In this case, first to Nth sub-calcification images SGI1 to SGIN corresponding to ultrasound images for each of the first to Nth sub-filter signals SFS1 to SFSN may be implemented. Thereafter, the sub-image display unit 410 may provide the final ultrasound calcification image UGI based on the correlation value between the first to Nth sub-calcification images SGI1 to SGIN. The method of generating a calcification image may be the same as the method of implementing a Doppler image, and more details may be as illustrated in FIGS. 10 and 11.

The detection device 10 for calcification according to the present disclosure transmits the transmitted ultrasound signal UT at the transmission frequency higher than the center frequency PF of the ultrasound probe, so that a low-band signal with high transmittance among the smoothed signals reflected from the calcification GM is included in the reception bandwidth of the ultrasound probe, thereby more accurately detecting the calcification GM within the object OB.

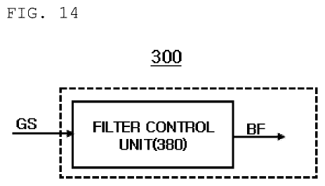

FIG. 12 is a diagram illustrating a frequency control unit included in the detection device for calcification of FIG. 1, FIG. 13 is a diagram illustrating a determination unit included in the detection device for calcification of FIG. 1, and FIG. 14 is a diagram illustrating a filter control unit included in the detection device for calcification of FIG. 1.

Referring to FIGS. 1 to 14, the band signal processing unit 300 may include the frequency control unit 310. The frequency control unit 310 may control a demodulation frequency DF for demodulating the received ultrasound signal UR.

In an embodiment, the frequency control unit 310 may include a determination unit 311. The determination unit 311 may determine the demodulation frequency DF according to the characteristics of the calcification signal GS of the received ultrasound signal UR. In addition, the band signal processing unit 300 may detect the calcification signal GS through color and power Doppler processing based on the received ultrasound signal UR. In another embodiment, the detection device 10 for calcification may display an ultrasound calcification image, which is generated based on the calcification signal GS on an ultrasound image using color.

The detection device 10 for calcification according to the present disclosure may include the frequency determination unit 100, the ultrasound transceiver unit 200, and the band signal processing unit 300. The frequency determination unit 100 may determine the transmission frequency TF of the transmitted ultrasound signal UT. The ultrasound transceiver unit 200 may transmit the transmitted ultrasound signal UT to the object OB and receive the received ultrasound signal UR reflected from the object OB. The band signal processing unit 300 may control the band of the received ultrasound signal UR to extract the calcification signal GS corresponding to the calcification included in the object OB.

In an embodiment, the transmission frequency TF may be controlled according to the frequency of the calcification signal GS within the band of the ultrasound probe included in the ultrasound transceiver unit 200.

In an embodiment, the center frequency of the bandpass filter 310 may be different from the transmission frequency of the transmitted ultrasound signal UT. In another embodiment, the frequency determination unit 100 may further include the transmission control unit 110. The transmission control unit 110 may control the transmission frequency based on the calcification signal GS.

In an embodiment, the band signal processing unit 300 may further include a filter control unit 380. The filter control unit 380 may control the center frequency of the bandpass filter 310 and the bandwidth of the bandpass filter 310 based on the calcification signal GS. In another embodiment, the transmission frequency of the ultrasound probe included in the ultrasound transceiver unit 200 may be controlled to detect the calcification signal GS.

According to present disclosure as described above, the following effects are provided.

The detection device for calcification according to the present disclosure transmits a transmitted ultrasound signal at a frequency within a band of the ultrasound probe and includes a band signal different from a transmitted signal frequency among smoothed signals reflected from the calcification in a reception bandwidth of the ultrasound probe, thereby more accurately detecting the calcification within the object.

In addition, other features and advantages of present disclosure may be newly identified through embodiments of present disclosure.

In addition to the technical problems of present disclosure described above, other features and advantages of present disclosure are described below, or can be clearly understood by those skilled in the art from such description and description.

What is claimed is:

1. A detection device for calcification, comprising:
a frequency determination unit that determines a transmission frequency of a transmitted ultrasound signal;
an ultrasound transceiver unit that transmits the transmitted ultrasound signal to an object and receives a received ultrasound signal reflected from the object; and
a band signal processing unit that controls the received ultrasound signal to extract a calcification signal corresponding to a calcification included in the object.

2. The detection device of claim 1, wherein a transmission frequency of an ultrasound probe included in the ultrasound transceiver unit is controllable to detect the calcification signal.

3. The detection device of claim 2, wherein the band signal processing unit includes a bandpass filter that filters the received ultrasound signal.

4. The detection device of claim 3, wherein a center frequency of the bandpass filter is different from the transmission frequency of the transmitted ultrasound signal.

5. The detection device of claim 4, wherein one end of a bandwidth of the bandpass filter is larger or smaller than the transmission frequency of the transmitted ultrasound signal.

6. The detection device of claim 5, further comprising:
an image display unit that provides an ultrasound calcification image based on the calcification signal.

7. The detection device of claim 2, wherein the frequency determination unit further includes a transmission control unit that controls the transmission frequency according to a resolution of the calcification included in the ultrasound calcification image.

8. The detection device of claim 7, wherein the band signal processing unit further includes a filter control unit that controls the center frequency of the bandpass filter and a bandwidth of the bandpass filter according to the resolution of the calcification included in the ultrasound calcification image.

9. The detection device of claim 3, wherein the band signal processing unit further includes a plurality of bandpass filters having a predetermined bandwidth.

10. The detection device of claim 9, further comprising:
a sub-image display unit that provides the ultrasound calcification image based on a correlation value between a plurality of sub-calculation images generated based on each of the sub-filter signals that filter the received ultrasound signal using the plurality of bandpass filters.

11. The detection device of claim 2, wherein the band signal processing unit includes a frequency control unit that controls a demodulation frequency that demodulates the received ultrasound signal.

12. The detection device of claim 11, wherein the frequency control unit includes a determination unit that determines the demodulation frequency according to the calcification signal of the received ultrasound signal.

13. The detection device of claim 1, wherein the band signal processing unit detects the calcification signal through color and power Doppler processing based on the received ultrasound signal.

14. The detection device of claim 1, wherein the detection device for calcification displays an ultrasound calcification image generated based on the calcification signal on an ultrasound image using color.

15. The detection device of claim 3, wherein the center frequency of the bandpass filter is different from the center frequency of the ultrasound probe.

16. The detection device of claim 1, wherein the frequency determination unit further includes a transmission control unit that controls the transmission frequency based on the calcification signal.

17. The detection device of claim 3, wherein the band signal processing unit further includes a filter control unit that controls the center frequency of the bandpass filter and the bandwidth of the bandpass filter based on the calcification signal.

18. The detection device of claim 1, wherein the transmission frequency is different from a center frequency of the ultrasound probe included in the ultrasound transceiver unit.
